(19) United States
(12) Patent Application Publication
(10) Pub. No.: US 2026/0141985 A1
(43) Pub. Date: May 21, 2026

(54) Method of Modeling Reaction Kinetics, Reaction Process, and Reactor System Using Deep Learning-Based Architecture

(71) Applicants: Zongxuan Hong, Houston, TX (US); Royce Hong, Houston, TX (US)

(72) Inventors: Zongxuan Hong, Houston, TX (US); Royce Hong, Houston, TX (US)

(21) Appl. No.: 18/952,008

(22) Filed: Nov. 19, 2024

(51) Int. Cl. G16C 20/10; G06N 3/044; G16C 20/70

(52) U.S. Cl. CPC G16C 20/10; G06N 3/044; G16C 20/70

(57) ABSTRACT

The present invention provides a method for modeling reaction kinetics and in reactor systems using deep learning techniques. The method involves modeling the reactor system as a series of incremental sections and configuring a deep learning model with a multi-layer neural network architecture to calculate reaction kinetics and process parameters within each section. The model further utilizes a recurrent neural network (RNN) to simulate the reaction process across sequential sections of the reactor, allowing for an accurate representation of reaction process along the reactor. The model is trained by comparing calculated results to actual physical measurements, optimizing the neural network's parameters and recurrent neural network's numbers and sizes. The invention enables compliance with physics principles, including conservation of mass and energy, and adapts to the complexities of industrial processes, comprising mass transfer, non-ideal flow dynamics, providing a method modeling catalytic reactions in industrial reactor systems.

### FIELD OF THE INVENTION

The present invention relates to the field of chemical reaction modeling and reactor process simulation. Specifically, the invention involves the application of deep learning techniques to model reaction kinetics and reaction process within a reactor system, wherein this invention employs a multilayer neural network to model individual sections of a reactor, while a recurrent neural network (RNN) is configured to represent the overall reactor system as a series of interconnected and sequential sections.

### BACKGROUND

Reaction kinetics is fundamental to the operation of industrial reaction processes, as it provides insight into the rates of chemical reactions and the factors affecting these rates. Understanding reaction kinetics allows industries to optimize process efficiency, yield, and safety. Traditionally, reaction kinetics has been derived using established mechanisms and kinetic theories, such as the Langmuir-Hinshelwood equation for gas-solid reactions involving solid catalysts. These classical methods offer valuable insights into the dynamics of chemical reactions under controlled conditions.

Beyond traditional approaches, more advanced kinetic modeling can be achieved through Microkinetics methods. Microkinetics involves the detailed study of the elementary steps of a reaction mechanism at the molecular level, providing a deeper understanding of the kinetics. This approach enables accurate, predictive modeling of complex reaction systems, leading to improved control over industrial processes.

However, both traditional and Microkinetics methods rely heavily on an in-depth understanding of the reaction mechanism and process involved. This requirement presents a significant challenge in industrial settings, where conducting such fundamental studies is often complicated to carry out or impractical. Industrial processes are typically complex, involving multiple reactions occurring simultaneously under varying conditions, making it even more challenging to apply these detailed kinetic studies.

In light of these challenges, there is a need for innovative methods that provide accurate and reliable reaction kinetics model without requiring extensive fundamental studies. The present invention introduces a novel approach that leverages deep learning techniques to address these issues, offering a practical and effective solution for determining reaction kinetics in industrial settings.

Developing accurate reaction kinetics models for industrial processes is particularly challenging due to numerous complex factors that are less encountered at laboratory scale. Industrial processes involve intricate heat and mass transfer phenomena that can significantly influence reaction rates and outcomes. To establish a reliable kinetics model, these factors must be carefully accounted for.

One of the main challenges in industrial processes is that industrial processes often operate in regimes where both intrinsic reaction rates and mass transfer rates control the overall reaction process. Unlike controlled laboratory conditions, industrial environments involve a complex interplay between chemical kinetics and mass transfer limitations, making it difficult to isolate and study these effects independently.

Additionally, industrial processes are characterized by complex flow dynamics, including flow channeling, non-uniform mixing, and both radial and axial flow variations. These flow dynamics can cause variations in reactant concentrations and temperature distributions within the reactor, further complicating the development of accurate kinetic models.

Another significant challenge is the much higher flow rates typically encountered at an industrial scale compared to laboratory conditions. These elevated flow rates can result in deviations from the reaction behaviors observed at smaller scales, requiring adjustments to the kinetics models to accurately reflect these differences.

Given these challenges, there is a critical need for innovative methodologies capable of addressing the complexities of industrial reaction kinetics. The present invention introduces a deep learning-based approach to effectively model reaction kinetics in industrial processes, overcoming the limitations of traditional methods and providing a robust solution for industrial applications.

To develop a kinetic model suitable for industrial processes, the proposed approach utilizes a deep learning model with several advanced features. This model employs neural networks designed to incorporate reaction mechanisms, reactor design, and complex factors such as heat and mass transfer, as well as the effects of high flow rates typically encountered in industrial environments. The deep learning model will be rigorously validated using physical property measurements and the fundamental principles of reactor design and thermodynamics to ensure its accuracy and reliability.

In an industrial process, operational limitations often lead to fewer variations and, consequently, fewer available data points or significant data variability. To overcome these challenges, it is essential to guide the machine learning model using fundamental scientific principles and prior knowledge of the reaction process. This guidance is crucial for appropriately constraining the model, ensuring that it produces accurate and reliable predictions. By incorporating these scientific fundamentals, the model can effectively learn from and generalize beyond the available data, resulting in a robust and reliable kinetic model suitable for industrial applications.

In prior arts, there are several research literatures related to applying deep learning approach to study chemical reactions, a few examples as mentioned in the following:

In the paper by Zhou, Z., Li, X., & Zare, R. N., “Optimizing Chemical Reactions with Deep Reinforcement Learning”, ACS Central Science, (2017) 3(12), 1337-1344, the authors introduce a novel application of deep reinforcement learning to optimize chemical reactions, focusing on finding the optimal sequence of transformations to improve target properties. The approach utilizes a deep Q-learning algorithm to navigate the chemical reaction space and identify optimal pathways to achieve desired products or reaction conditions. By treating chemical transformations as actions in a reinforcement learning environment, the model learns to maximize the reward associated with favorable reaction outcomes, such as higher yield or selectivity. This work represents an innovative combination of machine learning with chemical process optimization, providing a flexible method for discovering effective reaction sequences without requiring extensive human intervention or predefined rules. The demonstrated success of deep reinforcement learning in this context highlights its potential for automating reaction optimization in complex chemical environments.

In the paper by Xu, W., & Yang, B. “Microkinetic modeling with machine learning predicted binding energies of reaction intermediates of ethanol steam reforming: The limitations”, Molecular Catalysis, (2023), 537, 112940, the authors explore the use of machine learning for microkinetic modeling, specifically focusing on the prediction of binding energies of reaction intermediates in ethanol steam reforming. This study aims to enhance the microkinetic modeling process by integrating machine learning-predicted binding energies of key reaction intermediates, thereby facilitating a more efficient and data-driven approach to understanding ethanol steam reforming. The authors used machine learning techniques to predict the adsorption energies of intermediates, which play a crucial role in determining the reaction kinetics of catalytic processes. The study also highlights the limitations of using machine learning in this context, noting that the accuracy of predictions may vary depending on the training data quality and the complexity of the catalytic surface. The research provides valuable insights into the potential and challenges of leveraging machine learning for microkinetic modeling, especially in predicting reaction intermediates that are often difficult to obtain experimentally. This work demonstrates the opportunities and constraints of combining machine learning with catalytic modeling, contributing to the broader understanding of complex catalytic reaction mechanisms.

While there has been growing interest among researchers in using machine learning techniques to study chemical reactions, most of these efforts have focused on utilizing machine learning to explore reaction mechanisms using laboratory-scale data. Such research often involves relatively controlled conditions, which do not fully capture the complexities of reaction kinetics present in industrial settings. There have been limited applications of deep learning to study industrial chemical processes, where the complexity is significantly amplified by factors like mass transfer limitations, non-ideal flow dynamics, and the presence of multiple interacting phases. These challenges make it difficult to apply traditional or lab-scale models directly to industrial environments. Our invention aims to address these limitations by employing a deep learning-based approach tailored specifically for industrial chemical processes, providing robust modeling solutions capable of managing the intricate dynamics found in real-world industrial systems.

### SUMMARY OF THE INVENTION

The objective of this invention is to develop a deep learning-based method for modeling reaction kinetics and reaction processes specifically in complex industrial settings. Industrial chemical processes are inherently complicated, involving numerous factors such as intricate mass transfer, non-ideal flow dynamics, high flow rates, and interactions between multiple phases. These factors make conventional modeling approaches challenging to apply effectively. This invention leverages deep learning techniques to address these complexities, aiming to build accurate, data-driven models capable of capturing the intricate behavior of reaction kinetics under industrial conditions. By doing so, the invention seeks to provide a practical and reliable tool for predicting and optimizing industrial reaction processes, ultimately enhancing efficiency, yield, and operational safety.

Another objective of this patented method is to utilize advanced deep learning techniques to accurately model both reaction kinetics and the overall reaction process in complex industrial environments. Specifically, the method employs a multilayer neural network (NN) to model reaction kinetics within each defined reactor section, providing detailed insight into the behavior of chemical reactions at the local level. Additionally, the patented method incorporates a recurrent neural network (RNN) to represent the entire reactor system as a series of interconnected consecutive sections, effectively modeling the complete reaction process under real-world industrial conditions. This approach addresses the complexities of industrial processes, including intricate mass transfer, non-ideal flow dynamics, high flow rates, and interactions between multiple phases. By combining these neural network techniques, the invention provides a comprehensive modeling solution that is capable of managing the intricate dynamics and variability encountered in industrial reactor systems.

The present invention discloses a method for modeling reaction kinetics and reaction processes of a gas-solid and/or liquid-solid process in a reactor system containing at least one solid catalyst, the method comprising:
(a) modeling the reactor system as a series of continuous incremental sections extending from a reactor inlet to a reactor outlet, wherein the number and size of each incremental section is determined through a model training process;
(b) configuring a deep learning (DL) system with a multi-layer neural network (NN) architecture to compute reaction kinetics and processes within each incremental section, the neural network architecture comprising:
(i) an input layer configured to receive reaction process data, comprising information related to reactants and/or products, process parameters, operating conditions, reactor type and design, and environmental conditions as they enter the incremental section;
(ii) at least one sublayer configured to compute states of reaction intermediates and corresponding reaction rates, comprising surface intermediate species and/or transition state species, based on the received reaction process data;
(iii) an output layer configured to compute conversion rates of reactants and/or products, and associated process parameters;
(c) configuring a program to compute and check process data to comply with reactor design and physics principles, including conservation of mass and conservation of energy, for accurate representation of reaction process;
(d) configuring a recurrent neural network (RNN), wherein the multi-layer neural network forms a recurrent cell, the RNN being configured to model the reaction process across sequential incremental sections along the reactor, wherein each recurrent cell represents one incremental section and models the reaction process therein;
wherein,
(i) an input vector to each recurrent cell comprising reaction process data calculated from the preceding incremental section;
(ii) the recurrent cell applying the multi-layer neural network to compute reaction kinetics, reaction progress based on the input vector;
(iii) an output vector from each recurrent cell with process data passing to the subsequent recurrent cell corresponding to the next incremental section of the reactor;
(iv) the RNN iterating through all incremental sections of the reactor, terminating when the final recurrent cell reaches the outlet of the reactor, at which point the RNN outputs the overall reaction process results for the reactor system;
(e) training the deep learning model to optimize loss function by
(i) comparing calculated results from the RNN to actual physical measurements, comprising process data, product stream data, temperature, pressure where such measurements are available;
(ii) optimizing the comparison using a loss function with the objective of optimizing the neural network's weights, biases, and incremental section numbers and sizes in the RNN;
wherein the method is executed by at least one processing device comprising a processor coupled to a memory.

In one embodiment of the present invention, the reaction process involves reactants and products that can exist in either the gas phase, liquid phase, or a combination of both, while the catalyst remains in the solid phase. This embodiment allows for versatility in handling different types of reaction mixtures, ensuring compatibility with a wide range of industrial processes.

In another embodiment of the present invention, the reactor system comprises at least one reactor or a plurality of reactors arranged in series and/or in parallel. This configuration enables flexibility in reactor setup, allowing for optimized process control and scalability based on production requirements.

In a further embodiment of the present invention, the reactor employed in the system can be a plug flow reactor or a radial flow reactor, with the solid catalyst positioned stationary within the reactor.

In a further embodiment of the present invention, the reactor system may also operate in an adiabatic and/or isothermal mode, as described in the above embodiment. This feature provides flexibility in controlling temperature conditions, allowing the process to be adapted for optimal performance based on specific reaction kinetics and thermodynamic considerations.

In a further embodiment of the present invention, the configuration of the catalyst bed can be a fixed bed or a fluidized bed. This versatility allows the invention to address different reactor dynamics and provides the capability to select the most suitable catalyst arrangement for a particular industrial application.

In one embodiment of this invention, the reactor system is conceptualized by dividing it into incremental sections that can be represented as a continuous stirred tank reactor (CSTR). In this embodiment, the plug flow or radial flow reactor is simulated as a sequence of consecutive sections, where each section is considered uniform in terms of physical and chemical properties, comprising reactant and product concentrations, temperature, pressure, and reaction rates, except for the inlet conditions, which are passed from the preceding incremental section. This conceptualization allows for more efficient modeling and simulation of the reactor behavior, enhancing accuracy in predicting reaction kinetics under complex industrial conditions.

In a preferred embodiment of the present invention, the reaction kinetics and reaction process are modeled under steady-state or pseudo-steady-state conditions, wherein catalyst properties are assumed to be uniform and invariant throughout the process. The steady-state condition helps reduce the complexity of the deep learning model, thereby minimizing the computational burden associated with optimizing the neural network while ensuring accurate representation of the reaction kinetics and processes. By assuming that the catalyst properties remain constant, the model can focus on the essential aspects of the reaction mechanisms, which leads to a more efficient training process and accurate prediction of the reactor behavior.

In another preferred embodiment of the present invention, the reactor data is selectively used for periods during which the catalyst operates under steady-state conditions. These conditions are typically observed during the initial stages of fresh catalyst operation, when the catalyst properties are reasonably uniform throughout the reactor bed. By focusing on the steady-state phase, the model ensures that the input data is consistent and representative of uniform catalyst behavior, which facilitates more reliable training and accurate prediction of the reaction kinetics. This selective utilization of reactor data further contributes to reducing the computational demands of the model while maintaining accuracy.

In another preferred embodiment of the present invention, the reactor contains only a single type of catalyst with uniform catalytic properties throughout the reactor bed. The use of a single type of catalyst helps simplify the modeling process by reducing the number of variables that need to be considered, ensuring consistency across the reactor. This uniformity allows the model to more accurately represent the reaction kinetics and processes, as it does not need to account for variations in catalyst properties, thereby resulting in a more efficient deep learning model and improved prediction accuracy for the reactor system.

In one embodiment of the present invention, a kinetic modeling architecture for a reactor system is provided that employs a deep learning approach to accurately represent reaction kinetics and processes. The architecture features a multilayer neural network configured to model individual sections of the reactor system, capturing reaction kinetics within the section of the reactor. Additionally, a recurrent neural network (RNN) is employed to model the entire reactor system as a series of interconnected and sequentially arranged sections. The RNN structure allows for the representation of spatio and/or temporal dependencies and interactions between consecutive reactor sections, thereby providing a comprehensive model of the reactor system as a whole. The hierarchical relationship between the multilayer neural network, responsible for localized kinetics, and the RNN structure for sequential modeling, facilitates an accurate and dynamic understanding of the entire reactor system.

In another embodiment of the present invention, the deep learning model utilizes a multi-layer neural network architecture, including an input layer, at least one or a plurality of hidden layers, and an output layer, to perform mathematical calculations of reaction conversion rates for all relevant reaction species within a studied reactor section.

In another embodiment of the present invention, the input layer of the deep learning model receives an input vector that comprises process data, preferably based on prior knowledge of the reaction process. The prior knowledge can include information regarding the chemical reactants, operating conditions, and reactor design, allowing for a more informed representation of the initial state of the reaction.

In another embodiment of the present invention, the hidden layers of the deep learning model contain data related to, but not limited to, reaction intermediate species, transitional states, reaction temperature, pressure, and process rates. These hidden layers provide the necessary level of detail to accurately model the progression of the reaction, ensuring that the intermediate states are effectively captured and used to calculate the overall reaction kinetics.

In another embodiment of the present invention, the output layer of the deep learning model provides data related to, but not limited to, conversion rates of components in the product stream. This output information enables the model to quantify the extent of the reaction and assess the efficiency of the process, providing valuable insights into the reactor's performance and product formation.

In another embodiment of the present invention, the deep learning algorithm is configured such that the input vector is fed into the hidden layer, which consists of neurons that apply a linear transformation using a weight matrix with an added bias from a bias matrix, followed by a non-linear activation function to obtain the activated output, which is the hidden layer output. The hidden layer output is then fed into the output layer, which similarly consists of neurons applying a linear transformation with weights and biases, followed by a non-linear activation function to generate the activated output of the output layer. The non-linear activation function can include, but is not limited to, a Sigmoid, Tan h, ReLU, or a combination thereof, thereby allowing the model to capture complex non-linear relationships within the reaction processes.

In another embodiment of the present invention, the recurrent neural network (RNN) architecture utilizes a neural network model configured for a single reactor section as a recurrent cell, which is then applied sequentially to a series of consecutive cells to simulate the entire reactor system. By using the recurrent cell to model each incremental section of the reactor, the RNN can capture the progression of reaction kinetics and processes across the reactor length, thereby providing an accurate representation of the overall reactor behavior through sequential modeling of individual reactor sections.

In a preferred embodiment of the present invention, the number and size of the recurrent neural network cells can be varied during model training to more accurately represent the reaction process. One benefit is that by adjusting the number and size of the recurrent cells, the model can achieve a better balance between computational efficiency and accuracy. More importantly, the number and size of recurrent neural network cells along the reactor progression will be able to account for the complexities of industrial processes, including intricate mass transfer, non-ideal flow dynamics, high flow rates, and interactions between multiple phases. This unique approach enables the deep learning model to adapt to complex industrial processes for a more accurate representation of the reaction process throughout the reactor system.

In another embodiment of the present invention, the neural network and recurrent neural network are configured such that the output process data generated by the computation of each recurrent cell and the entire recurrent neural network complies with the physics principles of conservation of mass and conservation of energy. Compliance with these principles ensures that the model predictions adhere to fundamental physical laws, thereby improving the reliability of the output data.

In a preferred embodiment of the present invention, compliance with the principles of conservation of mass and conservation of energy is determined using methods that incorporate principles of reactor design and thermodynamics. By incorporating these methods, the model can effectively verify that the calculated reaction kinetics and processes are physically realistic, enhancing the accuracy and robustness of the predictions.

In a further embodiment of the present invention, the loss function of the deep learning model is a mathematical function configured to quantify the difference between the predicted output of the model and the actual physical measurements of process data, wherein the process data comprises parameters such as process gas compositions, concentrations, temperature, and pressure. The use of this loss function allows the model to iteratively refine its predictions by minimizing the error between calculated and measured values, thereby improving its predictive performance for reactor system behavior.

In one embodiment of the present invention, the training of the deep learning model involves optimizing the loss function through iterative adjustments of the model's parameters, including the weight matrix, bias matrix, and the number and size of recurrent neural network cells, to minimize the loss function. The optimization process employs gradient-based methods, such as stochastic gradient descent (SGD) and its variants, including Adam, RMSprop, and Adagrad, to calculate the gradients of the loss function with respect to the model parameters. These gradients are obtained using methods of propagation and/or backpropagation, enabling the model to iteratively update its parameters to converge on a solution that best represents the underlying reaction processes. By leveraging these advanced optimization techniques, the model can efficiently learn the complex relationships inherent in the reactor system data, resulting in a more accurate and robust representation of the reaction kinetics and overall process.

One advantage of the present invention is the use of neural networks with an intermediate hidden layer, which helps to capture detailed reaction kinetics based on the underlying reaction mechanisms, thereby providing a more intrinsic representation of the reaction kinetics.

Another advantage of the present invention is the use of matrix computation in deep learning method instead of the complicated ordinary differential equations typically employed in Microkinetics, making the model less complicated in terms of computation and optimization. This results in a reduction in computational complexity and an improvement in the efficiency of optimizing the deep learning model, ultimately leading to more accurate and reliable predictions of reactor behavior.

An additional advantage of the present invention is that it ensures compliance with principles of physics of conservation of mass and energy by incorporating knowledge of reactor design and thermodynamics, thereby enhancing the reliability, accuracy, and robustness of the model.

Another important and unique advantage of the present invention is the deep learning method employs a recurrent neural network with ability to vary the number and size of recurrent neural network cells during model training, allowing it to account for complex industrial processes such as mass transfer, non-ideal flow dynamics, high flow rates, and multiphase interactions, of which most current models will not be able to achieve.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further objects and advantages of the present invention will be apparent from the following detailed description of a presently preferred embodiment which is illustrated schematically in the accompanying drawings and programs.

### BRIEF DESCRIPTION OF THE FIGURES

The accompanying figures illustrate various embodiments of the principles described herein and are a part of the specification. The illustrated embodiments are merely examples and do not limit the scope of the claims. The elements in figures are not necessarily to absolute or relative definition. Further, the elements illustrated may have a variety of different configurations and/or representations.

FIG. 1 is a schematic representation of the kinetic modeling architecture for a reactor system employing a deep learning approach, wherein a multilayer neural network is used to model individual sections of the reactor system, and a recurrent neural network (RNN) is utilized to represent the entire reactor as a series of interconnected sections. The figure illustrates the hierarchical relationship between the multilayer neural network for localized kinetics and the RNN structure for sequential modeling of the entire reactor system.

FIG. 2 is a schematic representation of a multilayer neural network architecture, including its associated matrix representation for mathematical computation of the conversion rates of all relevant reaction species within a specific reactor section. The figure depicts the structure of the neural network used to model the kinetics of a reactor section, demonstrating how the input, hidden, and output layers are mathematically linked to perform calculations necessary for determining the reaction rates and subsequent conversions.

FIG. 3 is a schematic representation of a recurrent neural network (RNN) architecture that employs the neural network model developed for a single reactor section to represent the entire reactor system. The RNN structure captures the sequential nature of the reactor sections by using the output of each neural network cell as the input for the subsequent cell, thereby enabling a comprehensive study of the entire reactor system through a series of interconnected reactor sections.

FIG. 4 is a schematic representation of a computer programming algorithm for training the deep learning model to study reaction kinetics and reaction processes. The figure illustrates the workflow of the deep learning model training, including data input, model architecture setup, training iterations, and optimization of neural network parameters, such as weights and biases, and optimization of recurrent neural network, such as number and size of the cells, to accurately capture the underlying reaction dynamics.

Throughout the figures, identical reference numbers designate similar, but not necessarily identical, elements.

### DESCRIPTION OF A PREFERRED EMBODIMENT

Methods and systems that implement the embodiments of the various features of the invention will now be described with reference to the figures. The figures and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

In the following description, for purposes of explanation, numerous specific details are set forth in order to provide a thorough understanding of the present systems and methods. It will be apparent, however, to one skilled in the art that the present apparatus, systems and methods may be practiced without these specific details.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

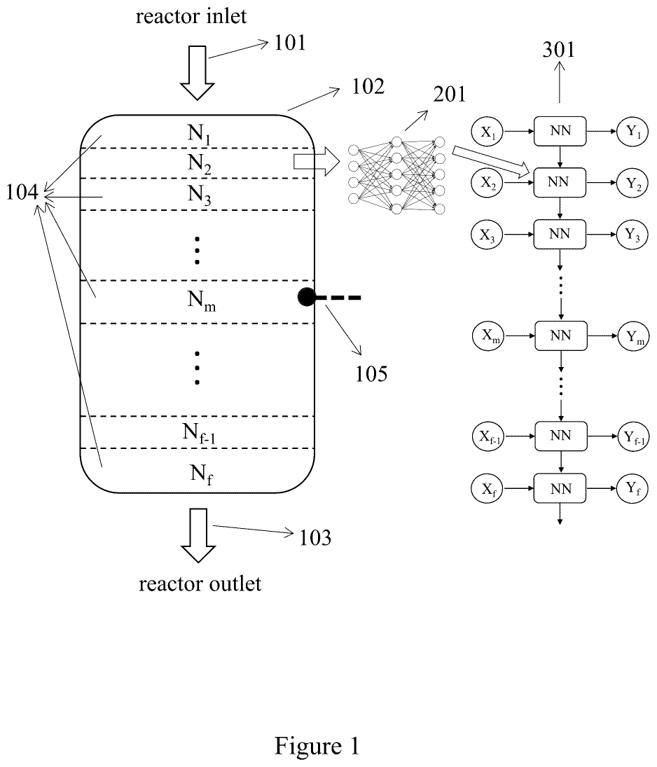

FIG. 1 illustrates a schematic representation of the kinetic modeling architecture for a reactor system, in accordance with the principles of reaction engineering and reactor design. In this invention, the reactor system is conceptualized as a series of reactor sections connected in sequence, with each section being treated as a continuously stirred tank reactor (CSTR). Each reactor section is assumed to exhibit uniform physical properties, such as concentration and temperature, under the assumption that all components are well-mixed. This approach allows for accurate modeling of reaction kinetics and simplifies the representation of complex reaction processes occurring throughout the entire reactor system.

The patented method utilizes a multilayer neural network (NN) to model reaction kinetics within each defined reactor section and employs a recurrent neural network (RNN) to represent the entire reactor system as a series of interconnected sequential sections.

Element 101 represents the reactor inlet, which typically comprises a feed stream with predefined inlet temperature and pressure. Element 102 denotes the reactor system as a whole. Element 103 represents the reactor outlet, which generally comprises a mixture of reactants and products in exiting stream, with temperature and pressure conditions resulting from the reaction process.

The reactor system is conceptualized as a series of reactor sections connected in sequence, as depicted by 104. In industrial processes, various physical parameters are measured along the length of the reactor system. For instance, at several specific positions along the reactor, reactor temperature and/or the chemical compositions of the process gas are measured, as exemplified by element 105 located at position Nm. Additionally, critical physical parameters are typically measured at the reactor outlet, such as the composition of the product stream, and the temperature and pressure of the product stream, as exemplified by element 106.

In the present invention, the reaction kinetics and reaction processes within each section of the reactor system are modeled using a deep learning method employing a multilayer neural network as depicted by Element 201, of which will be discussed in details accordingly. The entire reactor system is modeled by a recurrent neural network (RNN) as depicted by Element 301, of which will be discussed in details accordingly.

As a preferred embodiment, the reaction kinetics and reaction process under study are selected to be under steady-state or pseudo-steady-state conditions, wherein catalyst properties are assumed to be uniform and invariant throughout the process. This steady-state condition reduces the complexity of the deep learning model, thereby minimizing the computational burden associated with optimizing the neural network while ensuring accurate representation of the reaction kinetics. Given the limited availability and variability of commercial reactor data, a guided deep learning strategy is employed to enhance model robustness. The reactor data is selectively utilized for periods during which the catalyst operates at steady-state conditions, typically during the initial stages of fresh catalyst operation when catalyst properties can reasonably be assumed to be uniform throughout the reactor bed.

Furthermore, the reactor is a fix bed, adiabatic plug flow reactor.

Furthermore, there is only one type of catalyst inside the reactor which has the same catalytic properties.

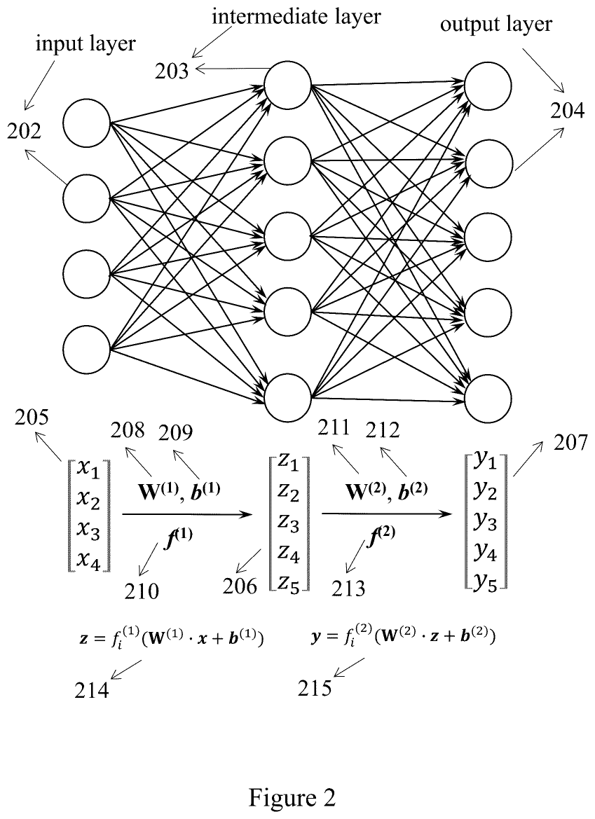

FIG. 2 is a schematic representation of the multilayer neural network architecture, along with the associated matrix representation for the mathematical calculations of reaction conversion rates for all relevant reaction species within a studied reactor section. Element 202 represents the input layer of the neural network, which comprises an array of data related to the incoming feed, such as feed components and concentrations. Feed flow rate, feed temperature and pressure are also used as input information. Element 203, representing hidden layers, comprise a plurality of the hidden layers, which may correspond to reaction intermediates and/or transitional states leading to product formation. The number of hidden layers used is based on model requirements and computation needs and capability. Element 204 represents the output layer, which comprise an array of calculated reaction rates for the formation of the relevant products.

Mathematically, the relationship between the input layer, hidden layer, and output layer of the neural network can be described as follows: the input layer receives a vector of concentrations for the relevant chemical species, represented by input vector x. This input vector is fed into the hidden layer, which consists of neurons that apply a linear transformation with bias, followed by a non-linear activation function. W⁽¹⁾ is the weight matrix connecting the input layer to the hidden layer, and b⁽¹⁾ is the bias vector. The resulting value is then passed through an activation function ƒ⁽¹⁾ to obtain the activated output, that is hidden layer output, represented by matrix z. The hidden layer output may represent the intermediates species and/or transitional state leading to formation of reaction products. The hidden layer output is subsequently fed into the output layer, which consists of neurons that apply another linear transformation with another bias, followed by another non-linear activation function. W⁽²⁾ is the weight matrix connecting the hidden layer to the output layer, and b⁽²⁾ is the bias vector. The resulting value is then passed through an activation function ƒ⁽²⁾ to obtain the activated output, the resulting output layer, represented by vector y, In this case, the output layer represents the rates of reaction for the corresponding chemical species in the product stream.

The mathematical operations are shown in the following equations.

$$x = [ \begin{matrix}{x}_{1} \\ {x}_{2} \\ {x}_{3} \\ {x}_{4}\end{matrix} ] \qquad ( Eq . 1 )$$

wherein x is the input vector with values xᵢ, such as x₁, x₂, x₃, x₄, as input values of inlet feed information.

$${W}^{( 1 )} = [ \begin{matrix}{w}_{11}^{( 1 )} & {w}_{12}^{( 1 )} & {w}_{13}^{( 1 )} & {w}_{14}^{( 1 )} \\ {w}_{21}^{( 1 )} & {w}_{22}^{( 1 )} & {w}_{23}^{( 1 )} & {w}_{24}^{( 1 )} \\ {w}_{31}^{( 1 )} & {w}_{32}^{( 1 )} & {w}_{33}^{( 1 )} & {w}_{34}^{( 1 )} \\ {w}_{41}^{( 1 )} & {w}_{42}^{( 1 )} & {w}_{43}^{( 1 )} & {w}_{44}^{( 1 )} \\ {w}_{51}^{( 1 )} & {w}_{52}^{( 1 )} & {w}_{53}^{( 1 )} & {w}_{54}^{( 1 )}\end{matrix} ] \qquad ( Eq . 2 )$$

wherein W⁽¹⁾ is the weight matrix connecting the input layer to the hidden layer.

$${b}^{( 1 )} = [ \begin{matrix}{b}_{1}^{( 1 )} \\ {b}_{2}^{( 1 )} \\ {b}_{3}^{( 1 )} \\ {b}_{4}^{( 1 )} \\ {b}_{5}^{( 1 )}\end{matrix} ] \qquad ( Eq . 3 )$$

wherein b⁽¹⁾ is the bias connecting the input layer to the hidden layer.

$${f}^{( 1 )} ( x ) = [ \begin{matrix}{f}^{( 1 )} ( {x}_{1} ) \\ {f}^{( 1 )} ( {x}_{2} ) \\ {f}^{( 1 )} ( {x}_{3} ) \\ {f}^{( 1 )} ( {x}_{4} )\end{matrix} ] \qquad ( Eq . 4 )$$

where in ƒ⁽¹⁾(x) is the activation function for input layer to the hidden layer.
There could be various types of activation as commonly used in Machine Learning.
For example, the activation function is a Sigmoid function, as shown in Eq. 5:

$${f}^{( 1 )} ( x ) = [ \begin{matrix}\frac{1}{1 + {e}^{- {x}_{1}}} \\ \frac{1}{1 + {e}^{- {x}_{2}}} \\ \frac{1}{1 + {e}^{- {x}_{3}}} \\ \frac{1}{1 + {e}^{- {x}_{4}}}\end{matrix} ] \qquad ( Eq . 5 )$$

$$z = [ \begin{matrix}{z}_{1} \\ {z}_{2} \\ {z}_{3} \\ {z}_{4} \\ {z}_{5}\end{matrix} ] \qquad ( Eq . 6 )$$

wherein z is the hidden layer output vector with values zᵢ, such as z₁, z₂, z₃, z₄, z₅, as output values for information about intermediate species and/or transitional states leading to corresponding species in product stream.
The hidden layer output is obtained by operations in Eq. 7;

$$z = {f}_{i}^{( 1 )} ( {W}^{( 1 )} \cdot x + {b}^{( 1 )} ) \qquad ( Eq . 7 )$$

Similarly, for the output layer,

$${W}^{( 2 )} = [ \begin{matrix}{w}_{11}^{( 2 )} & {w}_{12}^{( 2 )} & {w}_{13}^{( 2 )} & {w}_{14}^{( 2 )} & {w}_{15}^{( 2 )} \\ {w}_{21}^{( 2 )} & {w}_{22}^{( 2 )} & {w}_{23}^{( 2 )} & {w}_{24}^{( 2 )} & {w}_{25}^{( 2 )} \\ {w}_{31}^{( 2 )} & {w}_{32}^{( 2 )} & {w}_{33}^{( 2 )} & {w}_{34}^{( 2 )} & {w}_{35}^{( 2 )} \\ {w}_{41}^{( 2 )} & {w}_{42}^{( 2 )} & {w}_{43}^{( 2 )} & {w}_{44}^{( 2 )} & {w}_{45}^{( 2 )} \\ {w}_{51}^{( 2 )} & {w}_{52}^{( 2 )} & {w}_{53}^{( 2 )} & {w}_{54}^{( 2 )} & {w}_{55}^{( 2 )}\end{matrix} ] \qquad ( Eq . 8 )$$

wherein W⁽²⁾ is the weight matrix connecting the hidden layer to the output layer.

$${b}^{( 2 )} = [ \begin{matrix}{b}_{1}^{( 2 )} \\ {b}_{2}^{( 2 )} \\ {b}_{3}^{( 2 )} \\ {b}_{4}^{( 2 )} \\ {b}_{5}^{( 2 )}\end{matrix} ] \qquad ( Eq . 9 )$$

wherein b⁽²⁾ is the bias connecting the hidden layer to the output layer.

$${f}^{( 2 )} ( z ) = [ \begin{matrix}{f}^{( 2 )} ( {z}_{1} ) \\ {f}^{( 2 )} ( {z}_{2} ) \\ {f}^{( 2 )} ( {z}_{3} ) \\ {f}^{( 2 )} ( {z}_{4} ) \\ {f}^{( 2 )} ( {z}_{5} )\end{matrix} ] \qquad ( Eq . 10 )$$

where in ƒ⁽²⁾ (z) is the activation function for the hidden layer to the output layer.
There could be various types of activation as commonly used in Machine Learning.
For example, the activation function is a Sigmoid function, as shown in Eq. 11:

$${f}^{( 2 )} ( z ) = [ \begin{matrix}\frac{1}{1 + {e}^{- {z}_{1}}} \\ \frac{1}{1 + {e}^{- {z}_{2}}} \\ \frac{1}{1 + {e}^{- {z}_{3}}} \\ \frac{1}{1 + {e}^{- {z}_{4}}} \\ \frac{1}{1 + {e}^{- {z}_{5}}}\end{matrix} ] \qquad ( Eq . 11 )$$

$$y = [ \begin{matrix}{y}_{1} \\ {y}_{2} \\ {y}_{3} \\ {y}_{4} \\ {y}_{5}\end{matrix} ] \qquad ( Eq . 12 )$$

wherein y is the output layer vector with values yᵢ, such as y₁, y₂, y₃, y₄, y₅, as output values for information about species in product stream.
The output layer is obtained by operations in Eq. 13,

$$y = {f}_{i}^{( 2 )} ( {W}^{( 2 )} \cdot z + {b}^{( 2 )} ) \qquad ( Eq . 13 )$$

In a preferred embodiment of this invention, the output layer elements represent conversion rates to the corresponding species in the product stream.

Element 205 is a representative matrix for input layer, wherein each element of the matrix corresponds to related input value of the process, including but not limited to feed compositions of related species in the reaction, feed flow rate, feed temperature and pressure, catalyst property. Element 206 is a representative matrix for one hidden layer, wherein each element of the matrix could represent intermediate species and/or transitional state species to the formation of products, and associated physical properties of the process. Element 207 is a representative matrix for output layer, wherein each element of the matrix represents the reaction rates to a specific reactant and/or product.

Element 208 represents the weight matrix W⁽¹⁾ to the hidden layer. Element 209 represents the bias matrix b⁽¹⁾ to the hidden layer. Element 210 represents the activation function ƒ⁽¹⁾ for this layer. The mathematical relationship is showed in Eq. 7, as indicated in Element 214. Element 211 represents the weight matrix W⁽²⁾ to the output layer. Element 212 represents the bias matrix b⁽²⁾ to the output layer. Element 213 represents the activation function ƒ⁽²⁾ for this layer. The mathematical relationship is showed in Eq. 13, as indicated in Element 215.

To reduce model complexity and enhancing learning efficiency, prior knowledge is utilized to selectively identify and include relevant input variables within the input layer.

As discussed earlier, the section of reactor is conceptualized as a CSTR reactor wherein it is assumed all components are well-mixed, resulting in uniform physical properties, including but not limited to, concentrations, temperature and pressure throughout the section. And compositions of relevant chemical compounds in the feed stream are used in the input layer.

Input information such as, but not limited to, compositions of all components in the feed, feed flow rate, feed temperature, feed pressure are used in the calculation of the exiting stream properties that comply with physics principles of conservation of mass and conservation of energy, which is also referred as material balance and energy balance, accurately representing the underlying chemical processes.

Specifically, for a CSTR reactor approximation, the following equations are used to calculate corresponding information about the process based on knowledge of reactor design and thermodynamics to achieve material balance and energy balance.

As an example, the reaction process discussed here is considered in a steady state, and the reactor is an adiabatic reactor. Furthermore, for simplicity and the situation wherein the conversion is small and there is very small change in composition of gas streams, it is reasonable to assume the molar flow rate is close to a constant for inlet stream and outlet stream.

In the following calculations, all the units used are in molar, such as molar flow rate, molar concentration.

The correlation between mass flow rate and molar flow rate is by Eq. 14,

$${F}_{m a s s} = {F}_{m o l a r} \cdot M \qquad ( Eq . 14 )$$

wherein Fₘₐₛₛ is the mass flow rate, Fₘₒₗₐᵣ is the molar flow rate, and Mis the molecular weight of process gas,
wherein M can be an averaged molecular weight M_{avg} with process gas being a mixture of gases, obtained by Eq. 15,

$${M}_{a v g} = {\sum }_{i} {M}_{i} \cdot {x}_{i} \qquad ( Eq . 15 )$$

wherein Mᵢ is the molecular weight of species i, and xᵢ is the molar concentration of species i in the process gas stream.

For CSTR reactor design of a species in calculation, with assumption of steady state and no accumulation insider the reactor, is based on material balance of:
output of species=input of species+conversion of species within the section of reactor for a species A in Eq. 16,

$${F}_{output} {x}_{A , output} = {F}_{input} {x}_{A , input} + {r}_{A , current} {v}_{current section} \qquad ( Eq . 16 )$$

wherein Fₒᵤₜₚᵤₜ is the overall output molar flow rate, Fᵢₙₚᵤₜ is the overall input molar flow rate, x_{A,output} is the species A output concentration, x_{A,input} is the species A input concentration, r_{A,current} is the conversion rate of species A in the section of reactor under calculation, v_{current section} is the volume of reactor section under calculation, and Fₒᵤₜₚᵤₜ is assumed to be same value as Fᵢₙₚᵤₜ.

Based on this equation, with information of Fₒᵤₜₚᵤₜ, Fᵢₙₚᵤₜ, x_{A,input}, r_{A,current}, v_{current section} provided in the input information, x_{A,output}, is calculated by computer program.

Further, mass balance will comply with Eq. 17:

$${\sum }_{i} {F}_{output} \cdot {x}_{i , output} \cdot {M}_{i} = {\sum }_{i} {F}_{input} \cdot {x}_{i , input} \cdot {M}_{i} \qquad ( Eq . 17 )$$

In addition, the material balance of individual element of all compounds in the feed and product stream will comply with Eq. 18:

$${\sum }_{i} {F}_{output} \cdot {x}_{i , output} \cdot {N}_{i , C} = {\sum }_{i} {F}_{input} \cdot {x}_{i , input} \cdot {N}_{i , C} \qquad ( Eq . 18 )$$

wherein, N_{i,C} is the number of the specific element C, for instance, in the compound, and i represents all of the compounds in the feed and product stream. The Eq. 18 will be a series of element balances for all the elements in the feed stream.

For the energy balance, balance of enthalpy of all the compounds is evaluated. In the assumption of adiabatic reactor and reaction process at steady state, the total enthalpy of all compounds in the inlet feed stream is equal to total enthalpy of all compounds in the exit product stream, as shown in Eq. 19.

$${\sum }_{i} {F}_{output} \cdot {x}_{i , output} \cdot ( {H}_{i}^{Tout} - {H}_{i}^{0} ) = {\sum }_{i} {F}_{input} \cdot {x}_{i , nput} \cdot ( {H}_{i}^{Tin} - {H}_{i}^{0} ) \qquad ( Eq . 19 )$$

wherein, Hᵢ^{Tout} is the enthalpy value of compound i at temperature of outlet Tout, Hᵢ^{Tin} is the enthalpy value of compound i at temperature of inlet Tin, Hᵢ⁰ is the enthalpy value of compound i at standard reference temperature, for instance, reference temperature of 25° C., that is 298 degree Kelvin.

Enthalpy calculation of a compound follows an empirical equation Eq. 20.

$${H}_{i}^{T} = aT + \frac{b}{2} {T}^{2} + \frac{c}{3} {T}^{3} + \frac{d}{4} {T}^{4} \qquad ( Eq . 20 )$$

wherein, Hᵢ^{T} is the enthalpy value of compound i at temperature of T, a, b, c, d are constant values for a specific compound i, and the information are available in database of National Institute of Standards and Technology (NIST) of United States Department of Commerce.

Based on this equation, outlet temperature Tout is calculated and this will ensure compliance of energy balance for the section of the reactor under study.

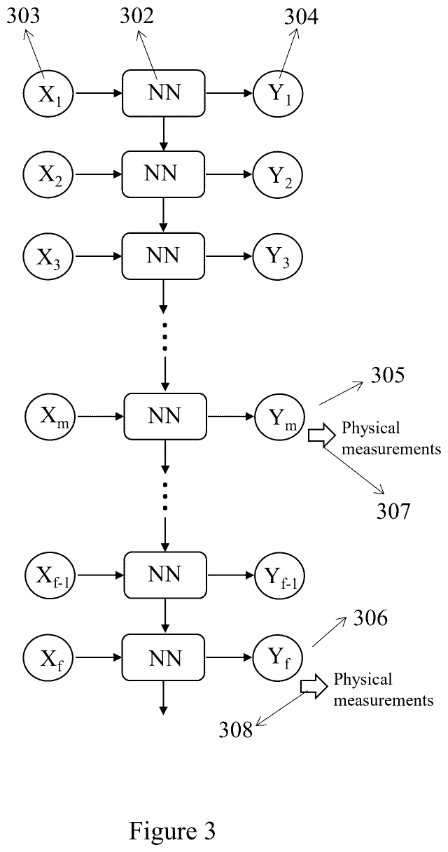

FIG. 3 is a schematic representation of the recurrent neural network (RNN) architecture, which employs the neural network model developed for a single reactor section to simulate the entire reactor system. The reactor system is modeled as a series of consecutive reactor sections, analogous to modeling a plug flow reactor (PFR) using a series of continuously stirred tank reactors (CSTRs), with each section represented as a CSTR model. The reaction kinetics within each section are determined by the neural network described previously, effectively capturing the complexities of the catalytic process. This neural network is then utilized as the recurrent cell within the RNN, sequentially applying it to each section of the reactor. The output from the output layer of each RNN cell, representing the reaction rates and associated calculations of reaction process information, is subsequently used as the input for the next RNN cell, thereby ensuring a consistent and continuous representation of reaction progression along the reactor system.

Element 302 represents the neural network described in FIG. 2. Element 303 represents the input matrix for an RNN cell, which contains the relevant data for the current reactor section, comprising feed composition, concentrations, temperature, and pressure. Element 304 represents the output matrix from the RNN cell, which includes calculated reaction rates and other relevant information. This output data is used to calculate the input information for the next consecutive RNN cell based on reactor design principles and the physical laws of mass and energy conservation. The details of calculation are described previously in FIG. 2 and associated neural network architecture. Along the reactor bed, there are locations where at least one physical parameter is measured, as exemplified by element 305 for RNN cell number m and element 307 for measured physical parameters. Additionally, at the reactor exit, critical physical parameters are measured, as exemplified by element 306 for the final RNN cell f and element 308 for corresponding physical measurements.

The comparison of the calculated values versus the measured values of these physical parameters establishes the loss function, which is used in model training. Eq. 21 is an example of loss function, specifically using mean squared error (MSE) to optimize the neural network weight matrix and bias matrix. Xₘ represents the input information passed on from preceding RNN cell output Yₘ₋₁, wherein the input information comprises process data.

$$M S E = \frac{1}{N} {\sum }_{i = 1}^{N} {( {y}_{i}^{measurement} - {y}_{i}^{calculation} )}^{2} \qquad ( Eq . 21 )$$

wherein MSE is mean squared error, i is the number of measurement, N is the total number of measurements, yᵢ^{measurement} is physical measurement value of i-th parameter, yᵢ^{calculationt} is calculated value of the corresponding parameter.

The total number of recurrent neural network (RNN) cell is determined by Eq. 22.

$$V = {\sum }_{m = 1}^{f} ( {v}_{m} ) \qquad ( Eq . 22 )$$

wherein V is the total reactor volume, m represents number on the RNN cell sequence, f represents the final number of RNN cell, and vₘ represents the volume of the section of the reactor on the m-th cell.

The vₘ value is used in the reactor design, material balance calculation. The model will continue to count the accumulated volume until it reaches the total reactor volume V value. In model training, the ƒ value and vₘ value can also be optimized in addition to weight matrix and bias matrix to reach the pre-defined error range of all measurements.

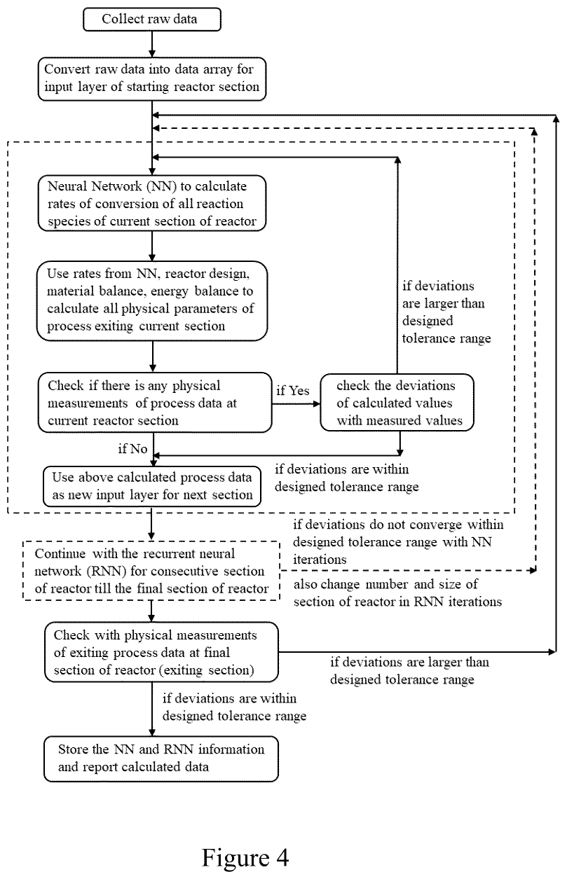

FIG. 4 is a schematic representation of a computer programming algorithm for training the deep learning model to study reaction kinetics and reaction processes. The figure illustrates the workflow of the deep learning model training, including data input, model architecture setup, training iterations, and optimization of neural network parameters, such as weights and biases, and optimization of recurrent neural network, such as number and size of the cells, to accurately capture the underlying reaction dynamics.

Basically, to train the model, the following steps are performed as in those corresponding flow diagram:
(1) Input data is collected from a data log file, and relevant data is fed into the input layer of the neural network.
(2) The neural network model then run as described earlier. At each recurrent neural network (RNN) cell, the database is checked to determine if there are any corresponding physical measurements. If no measurements are available, the model continues with the RNN. If measurements are available, a mean squared error (MSE) evaluation is performed to assess the difference between calculated values and physical measurements.
(3) If the error is within a predefined acceptable range, the model continues with the RNN. If the error exceeds the acceptable range, the process restarts to optimize the weight matrix and bias matrix of the neural network.
(4) The RNN continues sequentially, checking for physical measurements at each cell and repeating the process until the error falls within the predefined range.
(5) At the reactor exit, the final physical measurements are compared, and the MSE is further minimized to optimize the model's accuracy and performance.

### Example

The following example illustrates the application of the present invention to study the reaction kinetics and reaction process at an industrial scale for acetylene hydrogenation reaction. This example demonstrates the use of a deep learning approach for modeling a complex catalytic process at an industrial scale, wherein multiple factors such as large flow rates, flow dynamics, mass transfer and heat transfer, in addition to those common factors such as reactants concentrations, temperature and pressure, are all accounted for in the modeling. These factors are often challenging to investigate experimentally at the laboratory scale due to their inherent complexity and scale-specific effects. This example emphasizes the capability of the method to accurately capture the dynamic behavior of the reaction system, thereby providing valuable insights into reaction kinetics, reaction process in industrial reactors. Details of the method are described in the section of DESCRIPTION OF A PREFERRED EMBODIMENT.

The reaction process primarily involves the hydrogenation of acetylene (C₂H₂) with hydrogen (H₂) to form ethylene (C₂H₄), which is the principal target reaction (Eq. 23). A significant side reaction involves the hydrogenation of ethylene with hydrogen to form ethane (C₂H₆) (Eq. 24). Additionally, other side reactions include, but are not limited to, the oligomerization of acetylene and ethylene to form heavy hydrocarbons, such as C4+ long-chain alkenes, as well as carbon monoxide insertion reactions to form oxygenates. Since in this example, the reaction is assumed to be at initial stage of steady states, the side reactions were not counted in modeling.

$${C}_{2} {H}_{2} + {H}_{2} \to {C}_{2} {H}_{4} \qquad ( Eq . 23 )$$

$${C}_{2} {H}_{4} + {H}_{2} \to {C}_{2} {H}_{6} \qquad ( Eq . 24 )$$

This example describes an industrial ethane feed cracking process, wherein the cracked gas stream predominantly comprises hydrogen (H₂), carbon monoxide (CO), methane (CH₄), ethylene (C₂H₄), ethane (C₂H₆), and acetylene (C₂H₂). The commercial reactor utilized in this example is an adiabatic fix bed plug flow reactor with one type of acetylene hydrogenation catalyst. The feed stream is introduced into the reactor at a designed temperature (T) and pressure (P), thereby ensuring targeted conversion of acetylene.

Normally, in a commercial reactor, there are several specific locations along the reactor bed where physical parameters are measured. For instance, in the case of acetylene hydrogenation process in commercial reactors, reactor temperatures are measured at the inlet, ¼, ½, ¾, and exit positions along the reactor bed lengthwise by thermocouples inside the reactor. At the reactor exit, the compositions and concentrations, temperature, and pressure of the product stream are also measured. These measured parameters are critical for validating the deep learning model and ensuring an accurate representation of the reaction process.

It is known that the availability of commercial reactor data is limited, both in terms of quantity and variability, which poses challenges for effective model training. To address this, a guided deep learning strategy is employed to enhance model robustness. The modeling approach assumes that the catalyst and reaction system are at steady state, allowing for the assumption that catalyst properties remain uniform, which is reflected by maintaining the same weight matrix and bias matrix in the neural network. For this reason, the reactor data is selected only for the run time when the catalyst is considered operated at steady state and catalyst has no significant change in catalyst property. This type of run time is usually the initial period of fresh catalyst operation when it is reasonable to consider the catalyst properties are uniform throughout the reactor bed. Furthermore, prior knowledge is utilized to selectively identify and include relevant input variables within the input layer, thereby reducing model complexity and enhancing learning efficiency.

This exemplary model also integrates principles of reactor design and thermodynamics to calculate relevant process parameters, such as reaction temperature in reaction process, thereby ensuring the model adheres to fundamental physical laws and accurately represents the underlying chemical processes.

Based on prior knowledge, it is determined that hydrogen, carbon monoxide, ethylene, and acetylene are the primary components affected by the reaction, while other components in the feed have negligible effects. Therefore, only these affected components are selected as inputs to the input layer of the neural network model. Furthermore, it is assumed that only the concentrations of these components influence the reaction kinetics most within the section under study, with the consideration that temperature and pressure are close to constant values as the inlet values under the assumption of small conversion within the section of reactor. These concentration values are utilized as the input data in the input layer, allowing the model to focus on the most critical parameters that govern the reaction behavior, thereby improving the efficiency and accuracy of the deep learning model. Therefore, the input data is expressed as the following input matrix as Eq. 25.

$$x = [ \begin{matrix}{x}_{1} \\ {x}_{2} \\ {x}_{3} \\ {x}_{4}\end{matrix} ] \qquad ( Eq . 25 )$$

wherein x is the input vector, x₁ represents input concentration of H₂, x₂ represents input concentration of CO, x₃ represents input concentration of C₂H₂, x₄ represents input concentration of C₂H₄.

Based on theories of reaction mechanisms and Microkinetics, a hidden layer, here also referred to as the intermediates layer, is incorporated in the neural network. The values in hidden layer are regarded as precursors to the final product formation. The information represented in this hidden layer can be interpreted as corresponding to intermediate species adsorbed on the catalyst surface and/or transitional states of reactive species. The reactions involving these intermediate species and/or transitional states of reactive species directly determine the rates of formation for the reaction products, enabling the model to provide a mechanistic representation of the catalytic process that accounts for both the formation and consumption of key intermediates during the reaction pathway. In this example, the variable number in the hidden layer matrix is chosen to be the same as the number of products in the output layer, although it can be any number depending on prior knowledge of the reaction process and/or choice of model complexity. In the deep learning method of this invention, matrix-based mathematical computation replaces the use of ordinary differential equations in Microkinetics analysis, making computation more efficient.

Again based on prior knowledge of the process, the intermediate layer is expressed as the following hidden layer matrix in Eq. 26.

$$z = [ \begin{matrix}{z}_{1} \\ {z}_{2} \\ {z}_{3} \\ {z}_{4} \\ {z}_{5}\end{matrix} ] \qquad ( Eq . 26 )$$

wherein z is the hidden layer output vector, z₁ represents intermediate species of H₂*, z₂ represents intermediate species of CO*, z₃ represents intermediate species of C₂H₂*, z₄ represents intermediate species of C₂H₄*, z₅ represents intermediate species of C₂H₆*, of which C₂H₆ (ethane) is a byproduct of the reaction.

The output layer of the neural network represents the rates of reaction for the corresponding chemical compounds in the product stream. These calculated reaction rates are subsequently used in further calculations to determine the detailed concentration of chemical compounds in the product stream, based on the assumption of a continuously stirred tank reactor (CSTR) design and material balance requirement. In accordance with established physical laws, including material balance and energy balance, the reaction rates are applied to predict the concentrations and validate the consistency of mass and energy conservation throughout the reactor system. The outlet temperature is calculated from principles of thermodynamics. The use of reactor design and principles of physics in this deep learning model ensures the model calculation always comply with physics law of conservation of mass and energy.

Accordingly, the output data is expressed as the following output layer matrix as in Eq. 27.

$$z = [ \begin{matrix}{y}_{1} \\ {y}_{2} \\ {y}_{3} \\ {y}_{4} \\ {y}_{5}\end{matrix} ] \qquad ( Eq . 27 )$$

wherein y is the hidden layer output vector, y₁ represents conversion rate of H₂, y₂ represents conversion rate of CO, y₃ represents conversion rate of C₂H₂, y₄ represents conversion rate of C₂H₄, y₅ represents conversion rate of C₂H₆. Ethane is a byproduct of the reaction and it is part of component in the inlet feed, but based on prior knowledge, ethane has little effect on reaction mechanism.

The activation function used in this exemplary model is a Sigmoid function, as expressed in Eq. 5 and Eq. 11.

The entire reactor system is modeled as a sequence of consecutive reactor sections, akin to modeling a plug flow reactor (PFR) using a sequence of continuously stirred tank reactors (CSTRs), with each section represented by a CSTR model. The reaction kinetics within each section are determined by the neural network described earlier, which captures the complexities of the catalytic process. This same neural network is then employed as the recurrent cell within a recurrent neural network (RNN), effectively applying it to each section of the reactor in sequence. The output from the output layer of one RNN cell, which includes the rates of reaction, is used to determine the values for the input layer of the subsequent RNN cell, thereby ensuring a continuous and consistent representation of the reaction progression along the reactor.

The input data Xₘ fed to recurrent cell comprises input vector x to neural network and other process data including, but not limited to, flow rate (F), temperature (T), pressure (P), and concentrations of the rest of components not used in input vector x. The output data Yₘ form the recurrent cell comprises output vector y from neural network and other process data including, but not limited to, flow rate (F), temperature (T), pressure (P), and concentrations of the rest of components in the product stream. The data from Yₘ will feed into next recurrent cell Xₘ₊₁.

The detailed calculations for output Yₘ to ensure compliance with physics principles of mass conservation and energy conservation based on principles of reactor design and thermodynamics as shown in the following equations:

$${Fx}_{1 , m + 1} = {Fx}_{1. m} + {y}_{1 , m} {v}_{m} \qquad ( Eq . 28 )$$

$${Fx}_{2 , m + 1} = {Fx}_{2 , m} + {y}_{2 , m} {v}_{m} \qquad ( Eq . 29 )$$

$${Fx}_{3 , m + 1} = {Fx}_{3 , m} + {y}_{3 , m} {v}_{m} \qquad ( Eq . 30 )$$

$${Fx}_{4 , m + 1} = {Fx}_{4 , m} + {y}_{4 , m} {v}_{m} \qquad ( Eq . 31 )$$

$${Fx}_{5 , m + 1} = {Fx}_{5 , m} + {y}_{5 , m} {v}_{m} \qquad ( Eq . 32 )$$

wherein, F represents the inlet molar flow rate and it is assumed to be a constant throughout the reaction process based on prior knowledge that process gas compositions will have a very small change, therefore a very small change in the average molecular weight. vₘ is the volume of the section m in the sequences of CSTRs and recurrent cell m in recurrent neural network. Subscript number 1, 2, 3, 4, and 5 represent components H₂, CO, C₂H₂, C₂H₄, and C₂H₆ respectively. x_{1,m+1}, x_{2,m+1}, x_{3,m+1}, x_{4,m+1}, x_{5,m+1} represents the input vector for cell m+1 that is passed from preceding cell m output information of concentrations of those corresponding components. x_{1,m}, x_{2,m}, x_{3,m}, x_{4,m}, x_{5,m} represents the input vector for current cell m. y_{1,m}, y_{2,m}, y_{3,m}, y_{4,m}, y_{5,m} represents the output vector for current cell m, which represents the conversion rates of corresponding components.

The output results need to comply with material balance in Eq. 33 in that the total mass of output materials is equal to the total mass of input materials.

$${\sum }_{i ( m + 1 )} F \cdot {x}_{i ( m + 1 )} \cdot {M}_{i} = {\sum }_{i ( m )} F \cdot {x}_{i ( m )} \cdot {M}_{i} \qquad ( Eq . 33 )$$

wherein, i represents all the components in the process gas stream in addition to those calculated components of H₂, CO, C₂H₂, C₂H₄, and C₂H₆, however those components not calculated, such as CH₄ (methane), are essentially unchanged during the reaction process based on prior knowledge of the reaction process. And the molar concentration of all species i needs to be normalized to 1 for input and output values as in Eq. 34.

$${\sum }_{i , m + 1} {x}_{i , m + 1} = {\sum }_{i , m} {x}_{i , m} = 1 \qquad ( Eq . 34 )$$

The output results also need to comply with energy balance in Eq. 35 in that the total enthalpy of output materials is equal to the total enthalpy of input materials based on principles of thermodynamics.

$${\sum }_{i , m + 1} F \cdot {x}_{i , m + 1} \cdot ( {H}_{i}^{Tm + 1} - {H}_{i}^{0} ) = {\sum }_{i} F \cdot {x}_{i , m} ( {H}_{i}^{Tm} - {H}_{i}^{0} ) \qquad ( Eq . 35 )$$

wherein, i represents all the components in the process gas stream in addition to those calculated components, and those components not calculated, such as CH₄ (methane), will still have influence in the energy balance.

The detailed enthalpy calculations of all the components follows Eq. 20 with parameters for every component shown based on database of National Institute of Standards and Technology (NIST) of United States Department of Commerce.

$$For Hydrogen : a = 14.307 , b = 1.224 \times 1 {0}^{- 3} , c = - 0.163 \times 1 {0}^{- 6} , d = 0.014 \times 1 {0}^{- 9}$$

$$For Carbon Monoxide : a = 29.249 , b = - 0.191 \times 1 {0}^{- 3} , c = 0.400 \times 1 {0}^{- 6} , d = - 0.870 \times 1 {0}^{- 9} ,$$

$$For Acetylene : a = 46.024 , b = - 0.338 \times 1 {0}^{- 3} , c = 1.450 \times 1 {0}^{- 6} , d = - 1.771 \times 1 {0}^{- 9} ,$$

$$For Ethylene : a = 19.876 , b = 5.021 \times 1 {0}^{- 3} , c = - 0.487 \times 1 {0}^{- 6} , d = 0.121 \times 1 {0}^{- 9} ,$$

$$For Ethane : a = 4. 507 , b = 15.714 \times 1 {0}^{- 3} , c = - 0.725 \times 1 {0}^{- 6} , d = 0.052 \times 1 {0}^{- 9} ,$$

$$For Methane : a = 19.874 , b = 5.021 \times 1 {0}^{- 3} , c = - 0.487 \times 1 {0}^{- 6} , d = 0.121 \times 1 {0}^{- 9} ,$$

In recurrent neural network (RNN) calculations, the individual volume of each section of reactor, also corresponding to the recurrent cell size, will comply with Eq. 22, that is the addition of each cell size will equal to the total reactor volume.

In this example, the loss function uses mean squared error (MSE) to optimize the neural network weight matrix and bias matrix. Specifically, it is the measured temperature as program proceeds to those measured locations, and the measured acetylene concentration at the reactor exit location, which is one of the most important parameters. Eq. 36 shows the temperature loss function, and Eq. 37 shows the acetylene concentration loss function. And in particular, the requirement of range of error for Eq. 37 is pre-determined by requirement in compliance with plant setting.

$$M S E ( T ) = \frac{1}{N} {\sum }_{m}^{N} {( {T}_{m}^{measurement} - {T}_{m}^{calculation} )}^{2} \qquad ( Eq . 36 )$$

wherein, N is the number of temperature measurements along the reactor, Tₘ represents the temperature at the location of temperature measurement.

$$M S E ( {x}_{acetylene} ) = {( {x}_{f}^{measurement} - {x}_{f}^{calculation} )}^{2} \qquad ( Eq . 37 )$$

wherein, x_{f} represents the acetylene concentration at the reactor exit location.

The programing algorithm following the flow chart described in FIG. 4, wherein,
program subroutine (1) is the neural network for kinetics inside a section of reactor,
program subroutine (2) is the calculation of process data using principles of reactor design and thermodynamics for compliance of physics principles of conservation of mass and conservation of energy,
program subroutine (3) is the recurrent neural network to simulate the whole reactor,
program subroutine (4) is the error function at locations along the reactor bed and exit.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

What is claimed is:

1. A method for modeling reaction kinetics and reaction processes of a gas-solid and/or liquid-solid process in a reactor system containing at least one solid catalyst, the method comprising:
(a) modeling the reactor system as a series of continuous incremental sections extending from a reactor inlet to a reactor outlet, wherein the number and size of each incremental section is determined through a model training process;
(b) configuring a deep learning (DL) system with a multi-layer neural network (NN) architecture to compute reaction kinetics and processes within each incremental section, the neural network architecture comprising:
(i) an input layer configured to receive reaction process data, comprising information related to reactants and/or products, process parameters, operating conditions, reactor type and design, and environmental conditions as they enter the incremental section;
(ii) at least one sublayer configured to compute states of reaction intermediates and corresponding reaction rates, comprising surface intermediate species and/or transition state species, based on the received reaction process data;
(iii) an output layer configured to compute conversion rates of reactants and/or products, and associated process parameters;
(c) configuring a program to compute and check process data to comply with reactor design and physics principles, including conservation of mass and conservation of energy, for accurate representation of reaction process;
(d) configuring a recurrent neural network (RNN), wherein the multi-layer neural network forms a recurrent cell, the RNN being configured to model the reaction process across sequential incremental sections along the reactor, wherein each recurrent cell represents one incremental section and models the reaction process therein;
wherein,
(i) an input vector to each recurrent cell comprising reaction process data calculated from the preceding incremental section;
(ii) the recurrent cell applying the multi-layer neural network to compute reaction kinetics, reaction progress based on the input vector;
(iii) an output vector from each recurrent cell with process data passing to the subsequent recurrent cell corresponding to the next incremental section of the reactor;
(iv) the RNN iterating through all incremental sections of the reactor, terminating when the final recurrent cell reaches the outlet of the reactor, at which point the RNN outputs the overall reaction process results for the reactor system;
(e) training the deep learning model to optimize loss function by
(i) comparing calculated results from the RNN to actual physical measurements, comprising process data, product stream data, temperature, pressure where such measurements are available;
(ii) optimizing the comparison using a loss function with the objective of optimizing the neural network's weights, biases, and incremental section numbers and sizes in the RNN;
wherein the method is executed by at least one processing device comprising a processor coupled to a memory.

2. The reaction process of claim 1, wherein reactants and products are in gas phase and/or liquid phase, and catalyst is in solid phase.

3. The reactor system of claim 1, wherein the reactor system comprises at least one reactor or a plurality of reactors connected in series and/or in parallel.

4. The reactor system of claim 1, wherein the reactor is a plug flow type of reactor, or a radial flow type of reactor, wherein solid catalyst is placed stationarily inside the reactor.

5. The reactor system of claim 1, wherein the reactor is an adiabatic type of reactor, and/or an isothermal type of reactor.

6. The reactor system of claim 1, wherein the catalyst bed is a fixed bed, or a fluidized bed.

7. The incremental section of the reactor of claim 1, wherein the section of the reactor is conceptualized as a continuous stirred tank reactor (CSTR) and the plug flow reactor and/or the radial flow reactor are simulated by a consecutive sequence of sections of the reactor, wherein the section of reactor is considered uniform within the section body and in physical and chemical properties, comprising reactants and products concentrations, temperature, pressure, reaction rates, except the inlet conditions that is passed on from the preceding incremental section.

8. The process data of claim 1, wherein process data comprises physical and chemical properties of process gas, including, but not limited to, components and compositions, temperature, pressure, flow rate.

9. The input layer of claim 1, wherein input vector comprises process data, preferably, the relevant data based on prior knowledge of the reaction process.

10. The hidden layer of claim 1, wherein the hidden layer comprises data related to, but not limited to, reaction intermediate species and/or transitional states, reaction temperature, pressure, and rates of process.

11. The output layer of claim 1, wherein the output layer comprises data related to, but not limited to, conversion rates of components in the product stream.

12. The Deep Learning algorithm of claim 1, wherein the input vector is fed into the hidden layer, which consists of neurons that apply a linear transformation by a weight matrix with bias by a bias matrix, followed by a non-linear activation function to obtain the activated output, that is hidden layer output.

13. The Deep Learning algorithm of claim 1, wherein the hidden layer out is fed into the output layer, which consists of neurons that apply a linear transformation by a weight matrix with bias by a bias matrix, followed by a non-linear activation function to obtain the activated output, that is output layer data.

14. The Deep Learning algorithm of claim 1, wherein a non-linear activation function is, but not limited to, a Sigmoid, a Tanh, a ReLU, or a combination thereof.

15. The recurrent neural network of claim 1, wherein the architecture of the recurrent neural network (RNN) utilizes a neural network model configured for a single reactor section as a recurrent cell, and applies the recurrent cell sequentially to a series of consecutive cells to simulate the entire reactor system.

16. The neural network and recurrent neural network of claim 1, wherein the output process data generated by the computation of each recurrent cell and the entire recurrent neural network is configured to comply with the physics principles of conservation of mass and conservation of energy.

17. The neural network and recurrent neural network of claim 1, wherein the compliance with the principles of conservation of mass and conservation of energy is determined using methods comprising principles of reactor design and thermodynamics.

18. The loss function of claim 1, wherein the loss function is a mathematical function configured to quantify the difference between the predicted output of the model and the actual physical measurements of process data, wherein the process data comprises process gas compositions, concentrations, temperature, and pressure.

19. The training of deep learning model of claim 1, wherein optimizing the loss function comprises iterative adjustments of the model's parameters (weight matrix, bias matrix, recurrent neural network cell number and size) to minimize the loss function, wherein the optimization process employs gradient-based methods, including, but not limited to, stochastic gradient descent (SGD) and its variants, including Adam, RMSprop, and Adagrad, to calculate the gradients of the loss function with respect to the model parameters, wherein these gradients are obtained through methods of propagation and/or backpropagation.
